(19) United States
(12) Patent Application Publication
(10) Pub. No.: US 2025/0281889 A1
(43) Pub. Date: Sep. 11, 2025

(54) A PROCESS FOR STORING A TOLUENE DIISOCYANATE AND HIGH BOILERS CONTAINING MIXTURE AND A PROCESS FOR WORKING UP A TOLUENE DIISOCYANATE COMPRISING CRUDE REACTION PRODUCT

(71) Applicant: BASF SE, Ludwigshafen am Rhein (DE)

(72) Inventors: Stefan ENGELS, Ludwigshafen am Rhein (DE); Torsten MATTKE, Ludwigshafen am Rhein (DE); Tobias SUETSCH, Ludwigshafen am Rhein (DE)

(21) Appl. No.: 18/859,613

(22) PCT Filed: Apr. 25, 2023
(86) PCT No.: PCT/EP2023/060779

§ 371 Date: Oct. 24, 2024

(30) Foreign Application Priority Data
Apr. 26, 2022 (EP) 22170039.6

(51) Int. Cl. B01F 23/40; B01F 23/43; B01F 23/451; B01F 23/70; B01F 25/51

(52) U.S. Cl. CPC B01F 23/405; B01F 23/43; B01F 23/451; B01F 23/49; B01F 23/704; B01F 25/51; B01F 2215/0495

(57) ABSTRACT

The invention relates to a process for storing a toluene diisocyanate and high boilers containing mixture by feeding the toluene diisocyanate and high boilers containing mixture into a storage vessel (23) and agitating the toluene diisocyanate and high boilers containing mixture. The invention further relates to a process for working up a toluene diisocyanate comprising crude reaction product, comprising: (a) removing solvents from the toluene diisocyanate comprising crude reaction product (1) in a solvent removal (3), thereby obtaining a crude toluene diisocyanate (7); (b) removing toluene diisocyanate from the crude toluene diisocyanate (7) in a toluene diisocyanate removal (9), thereby obtaining purified toluene diisocyanate (11) as a product and a toluene diisocyanate comprising residue (13), or feeding the toluene diisocyanate comprising residue (13) into a hydrolysis in which the toluene diisocyanate is hydrolysed, thereby forming the respective toluene diamine; (c) feeding the toluene diisocyanate comprising residue (13) into a dryer (15) in which an essentially toluene diisocyanate comprising stream (17) and a dryer residue (19) are obtained; (d) recycling the essentially toluene diisocyanate comprising stream (17) into the toluene diisocyanate removal (9) in step (b); wherein at least part of the toluene diisocyanate comprising residue (13) is stored in a storage vessel (23).

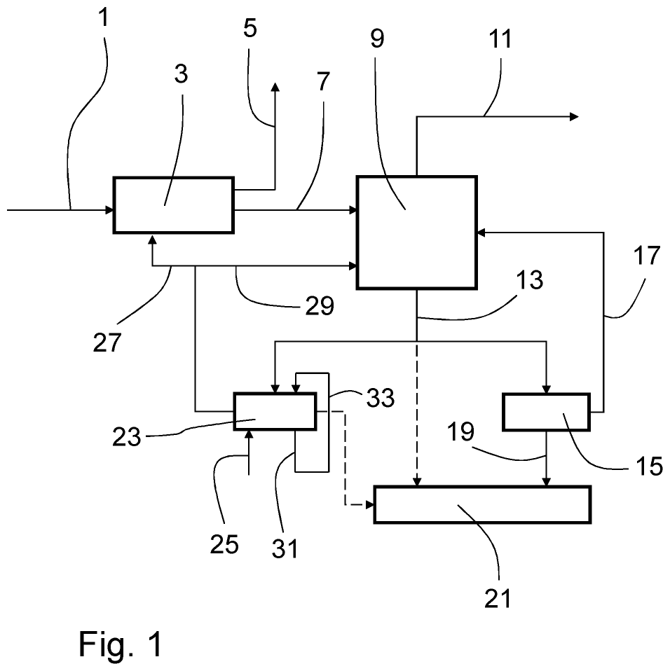

The invention relates to a process for storing a toluene diisocyanate and high boilers containing mixture, which contains 20 to 80% by weight of toluene diisocyanate. The invention further relates to a process for working up a toluene diisocyanate comprising crude reaction product by removing solvents from the toluene diisocyanate comprising crude reaction product, thereby obtaining a crude toluene diisocyanate, removing toluene diisocyanate from the crude toluene diisocyanate, thereby obtaining purified toluene diisocyanate as a product and a toluene diisocyanate comprising residue, feeding the toluene diisocyanate comprising residue into a dryer in which an essentially toluene diisocyanate comprising stream and a dryer residue are obtained, and recycling the essentially toluene diisocyanate comprising stream into the removing toluene diisocyanate from the crude toluene diisocyanate step.

Toluene diisocyanate (TDI) particularly is used as an intermediate product in the production of polyurethanes and usually produced by phosgenation of toluene diamine (TDA) in the gas phase or in the liquid phase.

The crude reaction product obtained by the phosgenation process contains impurities and is worked up for obtaining purified TDI as product. Working up the crude reaction product usually comprises at least one distillation step for removing low boilers like solvents and a further distillation step in which TDI as product is removed. In this distillation a distillation residue is obtained which still contains a remarkable amount of TDI.

For recovering the TDI from the distillation residue, a dryer may be used in which the TDI is evaporated and recycled into the distillation. The remaining dryer residue usually is thermally used, for example as a substitute of fuels like activated carbon in sewage sludge incineration or for the heating of cement kilns. Alternatively, the dryer residue may be decomposed for example in a hydrolysis process and recycled as TDA.

Processes for working up the distillation residue are described for example in DE-A 28 46 815, DE-A 29 15 830 or DE-A 43 17 669.

However, it is a disadvantage of all known processes that the distillation residue is highly viscous and that solids form during reprocessing, so that the plant components used for working up the distillation residue are subject to a certain risk of failure. As the distillation residue is not stable in storage and is subject to a constant increase in viscosity, presently the distillation residue is fed directly into the thermal use in case of such a failure. This direct use of the distillation residue in a thermal use results in remarkable losses of TDI. Further, it is necessary to provide incinerators of sufficient size.

For reducing the losses of TDI particularly if a dryer fails, an intermediate storage would be desirable until the plant parts which were shut down can be restarted.

Therefore, it is an object of the present invention to provide a process which allows for storing TDI containing mixtures. It is a further object of the present invention to provide a process for working up a toluene diisocyanate comprising crude reaction product, in which the losses of TDI are minimized.

This object is achieved by a process for storing a toluene diisocyanate and high boilers containing mixture, which contains 20 to 80% by weight of toluene diisocyanate, the process comprising:
(a) feeding the toluene diisocyanate and high boilers containing mixture into a storage vessel; and
(b) agitating the toluene diisocyanate and high boilers containing mixture.

Surprisingly it has shown that it is possible to store the TDI and high boilers containing mixture for a certain time, if the stored TDI and high boilers containing mixture is agitated, even though the general position is that this TDI and high boilers containing mixture is not stable in storage.

“Storing” in the context of the present invention means to deliver residence time without any goal to separate components of the feed mixture. This particularly means that during the time of storage separation between the TDI and the high boilers is as low as possible and, preferably, no separation between the TDI and the high boilers take place, and no intended reactions or evaporation of gaseous products takes place. However, changes in the composition due to adding higher or lower boiling components or due to time-related reactions, e.g. dimerization, trimerization of the TDI and/or reactions of other components in the mixture with each other which cannot be prevented are not excluded here and may have an influence on the viscosity of the stored mixture. Further, evaporation of low-boiling components like dissolved inert gases, reaction products such as carbon dioxide, due to the storage temperature also cannot be excluded, particularly when the temperature of the stored mixture changes. However, “storing” in the sense of the present invention does not include an intended evaporation and setting the temperature such that low boilers necessarily evaporate.

The TDI and high boilers containing mixture which can be stored using the inventive process contains 20 to 80% by weight of TDI, preferably 30 to 70% by weight and particularly 40 to 60% by weight and may be obtained for example as residue by working up a TDI containing crude reaction product. High boilers in the context of the present invention are all components having a boiling point above the boiling point of TDI. Such high boilers particularly comprise urea compounds, uretdiones, isocyanuric acid esters, carbodiimides, and higher condensed or polymerized compounds, particularly polymerized TDI.

Properties of the TDI and high boilers containing mixture like viscosity or component quantities in the TDI and high boilers containing mixture always relate to the complete mixture which may contain further components like low boilers or solvents besides the TDI and the high boilers.

During storage, particularly TDI polymerizes, which results in a viscosity increase of the TDI and high boilers containing mixture. By agitating, despite the viscosity increase, the TDI and high boilers containing mixture can be kept sufficiently fluid for a certain time. This allows for an intermediate storage and to feed the stored TDI and high boilers containing mixture into a further process step, particularly into a dryer at a later stage.

The storage period can be increased, if the viscosity of the TDI and high boilers containing mixture is reduced. Preferably, the viscosity of the TDI and high boilers containing mixture is set such that after a predefined storage time the viscosity is below 1 Pas and particularly preferably below 500 mPas. The viscosity is determined, for example by means of a rotational viscometer, capillary viscometer or inline by a tuning fork or other resonance frequency viscometer according to DIN 53019-1:2008, DIN 53019-2:2001 and DIN 53019-3:2008 or can be measured indirectly by e.g. power demand of agitator or circulation pump.

The viscosity of the TDI and high boilers containing mixture may be set before feeding into the storage vessel or, alternatively or additionally, in the storage vessel. The viscosity may be set for example by setting the content of solvent or TDI in the TDI and high boilers containing mixture or by setting the temperature. If the viscosity is set by adding TDI to the TDI and high boilers containing mixture, the amount of TDI added to the TDI and high boilers containing mixture preferably is selected such that the content of TDI in the TDI and high boilers containing mixture increases by at least 1% by weight, more preferred by at least 5% by weight and particularly by at least 10% by weight. If the viscosity is set by adding a solvent to the TDI and high boilers containing mixture, the amount of solvent added to the TDI and high boilers containing mixture is selected such that the content of TDI in the TDI and high boilers containing mixture does not fall below 20% by weight, preferably does not fall below 30% by weight and particularly does not fall below 40% by weight.

If solvent or TDI are used to set the viscosity of the TDI and high boilers comprising mixture in the storage vessel, the solvent or TDI may be introduced first into the storage vessel and subsequently the TDI and high boilers comprising mixture is fed into the storage vessel. Alternatively or additionally, it is also possible to feed the TDI and high boilers comprising mixture into the storage vessel and then add solvent or TDI. This further allows to additionally add solvent or TDI in case the viscosity increases during the storage time and reaches a predefined maximum value.

If a solvent is used for setting the viscosity, any solvent may be used in which TDI is soluble. Suitable solvents for example are selected from the group consisting of chlorinated aromatic hydrocarbons like dichlorobenzene, monochlorobenzene or trichlorobenzene or mixtures thereof, aromatic or aliphatic hydrocarbons like toluene, xylene, benzene, pentane, hexane, heptane, octane, cyclohexane, biphenyl, ketones such as 2-butanone, methyl isobutyl ketone, esters such diethyl isophthalate, ethyl acetate, butyl acetate, nitriles such as acetonitrile, sulfolane, and mixtures thereof. Particularly preferably, the solvent which is used for setting the viscosity of the TDI and high boilers comprising mixture is the same solvent as used in the production process of the TDI. Preferably, the solvent is monochlorobenzene, dichlorobenzene, trichlorobenzene, toluene or any mixture thereof, and particularly monochlorobenzene, dichlorobenzene or toluene.

Independently of setting the viscosity by adding a solvent or by adding TDI, if the solvent or the TDI is added before feeding the TDI and high boilers containing mixture into the storage vessel, the amount of solvent or TDI is selected such that the viscosity of the TDI and high boilers containing mixture is below 1 Pas, preferably below 500 mPas after a predefined storage time. The storage time preferably is selected such that it corresponds to a usual shutdown time of the dryer for maintenance, for example 1 to 10 days. However, when adding a sufficient amount of solvent and/or TDI, a storage time can be achieved which exceeds 10 days and may be in a range from 20 to 50 days or even longer. If the TDI and high boilers containing mixture and solvent are mixed in a ratio of 4:1, for example a storage time can be achieved which is about 20 days and if the mixing ratio of the TDI and high boilers containing mixture to the solvent is 1:1, a storage time of more than 50 days can be achieved.

Also when mixing the TDI and high boilers containing mixture and TDI in a ratio of 1:1, a storage time can be achieved which exceeds 50 days.

Alternatively, the viscosity of the TDI and high boilers containing mixture can be set by setting the temperature in the storage vessel. The lower the temperature in the storage vessel, the slower is the viscosity increase of the TDI and high boilers containing mixture. For setting the temperature, the TDI and high boilers containing mixture preferably is cooled to the intended temperature before feeding into the storage vessel. Additionally, for heating or cooling the TDI and high boilers comprising mixture which is stored in the storage vessel, the storage vessel may comprise a temper unit, for example a double jacket or tempering coils, and/or a heater cooler unit is provided in the external recirculation loop.

The temperature in the storage vessel preferably is in a range from 20 to 100° C. and particularly in a range from 35 to 85° C. To set the temperature in the storage vessel, any suitable device for setting the temperature can be used. Such devices for setting the temperature for example include storage vessels with a double jacket through which a heating medium flows or a heating coil in the storage vessel or on the outer wall of the storage vessel. The heating coil may be a pipe or tube through which a heating medium flows or an electrical heating coil. Further, any other device for setting the temperature known to a skilled person may be used like external heat exchangers or heaters in an external circuit. The heaters in the external circuit may be electrical heaters or heaters using a heating medium.

The pressure in the storage vessel preferably is in a range from 500 mbar (abs) to 2 bar (abs), particularly in a range from 800 mbar (abs) to 1.5 bar (abs).

For reducing or preventing fouling due to precipitation of high boilers, the TDI and high boilers containing mixture is agitated. By agitation, the TDI and high boilers containing mixture is kept in motion and due to this motion, a uniform distribution of the high boilers, which may be in the form of particles, highly viscous droplets or solved in the TDI and high boilers containing mixture, is achieved. Agitating of the TDI and high boilers containing mixture may be achieved by using a recirculation circuit through which the stored toluene diisocyanate is passed or by using an agitator in the storage vessel. If an agitator is used, any suitable stirrer by which the TDI and high boilers containing mixture is kept in motion can be used, for example axially conveying stirrers or radially conveying stirrers. Suitable stirrers for example are propeller mixer, paddle mixer or anchor stirrer.

If agitating the TDI and high boilers containing mixture is achieved by using a recirculation circuit, a stream of the TDI and high boilers containing mixture is withdrawn from the storage vessel, conveyed by a pump through the recirculation circuit and returned into the storage vessel. The pump used in the recirculation circuit can be any pump which is suitable for conveying viscous materials, for example a centrifugal pump, a displacer pump, a diaphragm pump or a canned motor pump.

Besides using an agitator or the recirculation circuit for agitating the TDI and high boilers containing mixture, it is also possible to provide both, the agitator and the recirculation circuit.

For reducing or preventing fouling, it is further preferred, if the storage vessel, connection lines to the storage vessel and further plant parts through which the TDI and high boilers containing mixture flows has no dead spaces, and, if the line for withdrawing the TDI and high boilers containing mixture from the storage vessel exits at the bottom of the vessel, the bottom is inclined in such a way, that the line for withdrawing the TDI and high boilers containing mixture is connected to the storage vessel at the lowest point of the storage vessel.

For pressure compensation during filling or emptying, the storage vessel may be connected to an inert gas supply. During emptying the storage vessel, inert gas is fed into the storage vessel and during filling the storage vessel with the TDI and high boilers containing mixture, inert gas is withdrawn from the storage vessel. In this way, by feeding inert gas into the storage vessel or withdrawing inert gas from the storage vessel, level variations may also be compensated. Further, it is preferred to flush the storage vessel with an inert gas before starting filling with the TDI and high boilers containing mixture into the storage vessel. For avoiding a reaction with components in the gas phase, it is further preferred to cover the TDI and high boilers containing mixture in the storage vessel with an inert gas phase. The inert gas used for pressure compensation, for flushing and/or for covering the TDI and high boilers containing mixture may be any gas which does not contain components which react with components of the TDI and high boilers containing mixture. Suitable inert gases for example are nitrogen, noble gases like argon or carbon dioxide. Particularly preferably, the inert gas is nitrogen. If the storage vessel is connected to an inert gas supply it is further preferred to use the inert gas to set the pressure in the storage vessel.

To avoid gas being directly introduced into the atmosphere, the storage vessel further may be connected to an exhaust gas system or filter system in which gas withdrawn from the storage vessel is purified before being introduced into the atmosphere.

The TDI and high boilers containing mixture which is stored in the storage vessel may be any mixture which comprises TDI and high boilers. However, preferably, the TDI and high boilers containing mixture is a TDI comprising residue which is obtained by working up a TDI comprising crude reaction product.

The process for working up the TDI comprising crude reaction product preferably comprises:
(a) removing solvents from the toluene diisocyanate comprising crude reaction product in a solvent removal, thereby obtaining a crude toluene diisocyanate;
(b) removing toluene diisocyanate from the crude toluene diisocyanate in a toluene diisocyanate removal, thereby obtaining purified toluene diisocyanate as a product and a toluene diisocyanate comprising residue;
(c) feeding the toluene diisocyanate comprising residue into a dryer in which an essentially toluene diisocyanate comprising stream and a dryer residue are obtained and recycling the essentially toluene diisocyanate comprising stream into the toluene diisocyanate removal in step (b), or feeding the toluene diisocyanate comprising residue into a hydrolysis in which the toluene diisocyanate is hydrolysed, thereby forming the respective toluene diamine;
According to the present invention, at least part of the toluene diisocyanate comprising residue is stored at least temporary in a storage vessel.

The TDI comprising crude reaction product may be obtained by any suitable process for producing TDI known to a skilled person. Usually, TDI is formed by reacting toluene diamine (TDA) and phosgene in the presence of solvents. Thus, the TDI comprising crude reaction product obtained in the reaction additionally contains phosgene, solvent, hydrochloride and secondary products which also form in the reaction. For working-up the crude reaction product, in a first step the non-reacted phosgene and the hydrochloride are removed. The TDI comprising crude reaction product which is worked-up by the process of this invention is the composition which is obtained after removing the phosgene and hydrochloride.

Removing the solvent in step (a) and the TDI in step (b) may be carried out as described in DE-A 102 60 092.

For working-up, in a first step (a), solvents are removed from the TDI comprising crude reaction product, thereby obtaining a crude TDI. Preferably, the solvents are removed by a first distillation process. The first distillation process may comprise one distillation stage or more than one distillation stages, for example two or three distillation stages. If the first distillation process is carried out in more than one distillation stage, usually each distillation stage is carried out in a separate distillation column. Usually, the solvents are low boilers, which means that the boiling point of the solvents is below the boiling point of the TDI. For this reason, if the distillation is carried out in a distillation column, the solvents are removed at the top of the distillation column and the crude TDI is removed at the bottom of the distillation column. The crude TDI contains high boilers and remaining solvent. The first distillation process for removing the solvents from the TDI comprising crude reaction product preferably is carried out at a bottom temperature of 90 to 260° C., more preferred at a bottom temperature of 120 to 220° C. and particularly at a bottom temperature of 150 to 200° C. and a pressure in the range from 2 to 1000 mbar (abs), preferably in a range from 50 to 200 mbar (abs). If the first distillation process is carried out in more than one distillation column, usually the distillation columns are operated at stepwise decreased pressure within the above ranges.

From the thus obtained crude TDI, purified TDI is removed as product in step (b). Removing the TDI from the crude TDI preferably also is carried out by distillation. In this second distillation, the purified TDI obtained as product may be obtained as top stream, or, if further low boilers are removed in the second distillation as a side stream. If the purified TDI is obtained as a side stream, the further low boilers are removed as top stream. In each case, the bottom stream of the second distillation is the TDI comprising residue. This TDI comprising residue still may contain 20 to 80% by weight of TDI, preferably 40 to 60% by weight of TDI. The second distillation process for removing the purified TDI from the crude TDI preferably is carried out at a bottom temperature of 90 to 260° C., more preferred at a bottom temperature of 100 to 200° C. and particularly at a bottom temperature of 110 to 150° C. and a pressure in the range from 2 to 1000 mbar (abs), preferably in a range from 5 to 50 mbar (abs). The second distillation may comprise one distillation stage or more than one distillation stages, for example two or three distillation stages. A distillation in a divided wall column is also possible. If the second distillation is carried out in more than one distillation stage, usually each distillation stage is carried out in a separate distillation column.

The TDI comprising residue is fed into a dryer in step (c) for recovering the TDI which is obtained in the TDI comprising residue and thus increasing the yield of the process or alternatively into an hydrolysis unit in which the TDI is hydrolysed, thereby forming the respective toluene diamine (TDA). The essentially TDI comprising stream preferably is recycled into step (b), in which the purified TDI is removed from the crude TDI. The dryer residue which particularly comprises highly viscous or even solid components, usually is fed into a thermal use, for example as a substitute of fuels like activated carbon in sewage sludge incineration or for the heating of cement kilns. Alternatively, the dryer residue can be fed into a hydrolysis, in which the TDI can be decomposed and recycled as TDA.

The dryer may be any suitable dryer, particularly a mechanical direct dryer, for example an extruder dryer, a paddle dryer, a fluidized bed dryer or a thin film dryer.

Further, the dryer may comprise a fluidized bed as described for example in DE-A 29 15 830.

Separating the TDI from the TDI comprising residue usually is carried out at a temperature in a range from 100 to 350° C., more preferred in a range from 150 to 300° C. and particularly in a range from 180 to 270° C. and a pressure in a range from 5 to 300 mbar (abs), more preferred in a range from 10 to 200 mbar (abs) and particularly in a range from 20 to 150 mbar (abs).

According to the invention, at least a part of the TDI comprising residue obtained in step (b) is stored in a storage vessel.

For storing the at least part of the TDI comprising residue, it is possible to continuously branch off a part of the TDI comprising residue and feed this branched off part into the storage vessel. However, preferably, the TDI comprising residue only is fed into the storage container, when the dryer or hydrolysis is not operating, for example if the dryer has a failure and/or is shut down. By feeding the TDI comprising residue into the storage container, it is possible to regain the contained TDI at a later point when the dryer is in operation again. For this reason the yield can be remarkably increased as according to the present state of the art, in case the dryer is not in operation, the whole TDI comprising residue is incinerated.

Particularly in case the dryer or hydrolysis is not in operation, the whole TDI comprising residue obtained in step (b) is fed into the storage vessel until the storage vessel is completely filled. After being completely filled, also in the inventive process, it is necessary to remove the TDI comprising residue from the process, and feed it into the thermal use. However, preferably, the storage vessel is dimensioned such, that the whole TDI comprising residue, which is obtained during a usual shut-down period of the dryer, can be fed into the storage vessel.

As described above, the storage period can be increased if the viscosity of the TDI comprising residue is reduced. Preferably, the viscosity of the at least part of the TDI comprising residue which is stored in the storage vessel is set such that after a predefined storage time the viscosity is below 1 Pas and particularly below 500 mPas and may be set for example by setting the content of solvent or by the content of TDI in the TDI comprising residue or by setting the temperature in the storage vessel.

Besides the above described method for setting the viscosity by adding TDI for setting the content of TDI in the TDI comprising residue, in the inventive process for working-up the TDI comprising crude reaction product, it is also possible to set the viscosity of the at least part of the TDI comprising residue by setting the amount of TDI in the TDI comprising residue by removing of TDI in step (b). This can be achieved for example by reducing the amount of purified TDI withdrawn from the apparatus in which step (b) is carried out. If a distillation column or an evaporator is used for removing the TDI from the from the crude TDI, this can be achieved for example be reducing the bottom temperature or by reducing the evaporation temperature, respectively.

To avoid precipitation of high boilers contained in the TDI comprising residue, it is preferred to agitate the TDI comprising residue in the storage vessel. Agitating may be carried out as described above by using an agitator for stirring the TDI comprising residue in the storage vessel and/or by connecting the storage vessel to a recirculation circuit through which the stored TDI comprising residue is passed.

In the process for working up the TDI comprising crude reaction product, the recirculation circuit may be a separate circuit or part of the piping which is used for conveying the TDI comprising residue from the storage vessel back into the process for working-up the TDI.

If the piping which is used for conveying the TDI comprising residue is used as recirculation circuit, usually an additional connection is necessary through which the TDI comprising residue can be fed from the line through which the TDI comprising residue is withdrawn from the storage vessel back into the storage vessel. For this purpose, preferably a connecting line is provided which connects the line through which the TDI comprising residue is withdrawn from the storage vessel with the storage vessel. To allow the TDI comprising residue being recycled into the process or to remove TDI comprising residue from the process, it is preferred that the connecting line comprises a control element for closing the connecting line, for example a valve, a tap or a stopcock. Further, for recirculating the TDI comprising residue for agitating, at least one further control element for closing a line is provided in the line through which the TDI comprising residue is withdrawn upstream the connecting point of the connecting line.

By closing the control element in the line for withdrawing the TDI comprising residue and opening the control element in the connecting line, the TDI comprising residue is recirculated from the storage vessel through the line for withdrawing the TDI comprising residue and the connecting line back into the storage vessel and by closing the control element in the connecting line and opening the control element in the line for withdrawing the TDI comprising residue, the TDI comprising residue can be withdrawn and either be recycled into the work up process or withdrawn from the process and, for example, being fed into a thermal use. After use of storage vessel the storage vessel has to be cleaned by flushing with TDI or solvent to avoid plugging or fouling.

An embodiment of the inventive process for working up a TDI comprising crude reaction product is shown in the only FIGURE.

The FIGURE shows schematically a process for working up a TDI comprising crude reaction product.

For working up a TDI comprising crude reaction product 1, the crude reaction product 1 is fed into a solvent removal 3. In the solvent removal 3, solvent 5 is removed from the crude reaction product 1 preferably by distillation or by evaporation. The solvent 5 usually is a low boiler, so that TDI and high boilers form one fraction and the solvent a second fraction. The solvent 5 removed from the TDI comprising crude reaction product can be recycled into the production process for TDI, if applicable after further working up by which low boiling impurities which also may be removed in the solvent removal 3 are removed from the solvent before recycling the solvent into the production process.

The TDI and high boilers comprising second fraction is withdrawn as crude TDI 7 from the solvent removal 3 and fed into a TDI removal 9. In the TDI removal, a part of the TDI is separated from the crude TDI and withdrawn from the TDI removal as purified TDI 11, which is the product of the work up process.

Remaining TDI and high boilers form a TDI comprising residue 13, which also is withdrawn from the TDI removal 9. During normal work-up process, the TDI comprising residue 13 is fed into a dryer 15. In the dryer 15, TDI and optionally further components which evaporate in the dryer are removed and recycled into the TDI removal 9 as recovered TDI 17. As an alternative, the TDI comprising residue 13 may be fed into a hydrolysis in which the TDI is hydrolysed, thereby forming the respective TDA. The TDA then may be worked-up for removing water and recycled into the reaction forming the TDI.

All components which are not evaporated in the dryer 15 form a dryer residue 19, which is removed from the dryer 15 and usually fed into a thermal use 21. Besides a thermal use 21, the dryer residue also may be fed into any other use. However, usually, the dryer residue is incinerated for energy generation, used as a substitute for activated carbon in the sewage sludge incineration or for heating cement kilns. Alternatively, the dryer residue may be decomposed, for example in a hydrolysis process, and recycled as TDA.

According to the invention, during normal work-up of the TDI comprising crude reaction product, a part of the TDI comprising residue 13 may be fed into a storage vessel 23. However, preferably, during normal operation the whole amount of the TDI comprising residue 13 is fed into the hydrolysis or dryer 15 and only in case of a shutdown of the hydrolysis or dryer 15, for example for maintenance purposes or during a possible malfunction of the hydrolysis or dryer, the whole TDI comprising residue 13 which is formed in the TDI removal 9 is fed into the storage vessel 23.

In order to be able to further process the TDI comprising residue 13 which is stored in the storage vessel 23 after restarting the dryer or hydrolysis, for example after finishing the maintenance or after resolving the malfunction, it is necessary to keep the TDI comprising residue 13 in a flowable condition and further to avoid precipitation of components from the TDI comprising residue 13, particularly of high boilers which may form solids.

For keeping the TDI comprising residue 13 in a flowable condition, preferably the viscosity of the TDI comprising residue is set such that it is below 1 Pas, preferably below 500 mPas. For setting the viscosity of the TDI comprising residue, it is for example possible to set the content of solvent or TDI in the TDI comprising residue 13. For setting the content of solvent, solvent may be fed into the storage vessel 23 via a feed line 25. Accordingly, the viscosity can also be set by adding TDI via the feed line 25, if it is intended to set the viscosity by the amount of TDI in the TDI comprising residue. If the viscosity is set by the amount of TDI in the TDI comprising residue, it is alternatively or additionally also possible to operate the TDI removal 9 in such a way that the amount of purified TDI 11 is reduced and such the amount of TDI in the TDI comprising residue 13 is increased. This may be realized by reducing the temperature or increasing the pressure in the TDI removal 9, if the TDI removal 9 comprises a distillation or evaporation.

If solvent or TDI are used to set the viscosity of the TDI comprising residue in the storage vessel 23, the solvent or TDI may be introduced first into the storage vessel 23 and subsequently the TDI comprising residue 13 is fed into the storage vessel 23. Alternatively or additionally, it is also possible to feed the TDI comprising residue 13 into the storage vessel 23 and then add solvent or TDI via the feed line 25. This further allows to additionally add solvent or TDI in case the viscosity increases during the storage time and reaches a predefined maximum value. However, preferably, first the solvent or TDI are introduced into the storage vessel 23 and subsequently the TDI comprising residue 13.

Alternatively or additionally, the viscosity of the TDI comprising residue in the storage vessel 23 may be set by setting the temperature in the storage vessel. The lower the temperature in the storage vessel 23, the slower is the reaction of the components in the TDI comprising residue forming high boiling components, like oligomers or polymers. For this reason, the viscosity of the TDI comprising residue in the storage vessel 23 increases slower than at a higher storage temperature.

For reducing precipitations from the TDI comprising residue, it is preferred to agitate the TDI comprising residue in the storage vessel 23. For this purpose, it is possible to provide a stirrer in the storage vessel 23. By stirring with the stirrer, the TDI comprising residue in the storage vessel 23 is kept in motion and by this all components in the TDI comprising residue are kept in a largely uniform mixture.

As an alternative or an addition to the stirrer, also a recirculation circuit 31 can be provided for agitating the TDI comprising residue. Such a recirculation circuit 31 may be a separate circuit or part of the piping of the apparatus for working up the TDI comprising crude reaction product.

If the recirculation circuit 31 is a separate circuit, a recirculation line 33 is connected to the storage vessel 23, particularly at the bottom of the storage vessel 23 and ends in the storage vessel 23, for example at a side entry into the storage vessel 23 or at the top of the storage vessel 23. For recirculating the TDI comprising residue, the recirculation line is equipped with a pump. The pump may be any pump by which the TDI comprising residue can be conveyed, for example a centrifugal pump, a displacer pump, a diaphragm pump or a canned motor pump.

As an alternative to a separate recirculation line, the recirculation circuit also may be realized by using the piping of the apparatus. In this case, for recirculating, the TDI comprising residue may be withdrawn from the storage vessel 23 either by the line through which the TDI comprising residue is recycled into the process or by the line through which the TDI comprising residue is fed into the thermal use 21. From the respective line which is used for recirculation, a connecting line branches and ends in the storage vessel 23. By closing the respective withdrawal line upstream the branching point of the connecting line and opening the connecting line, the TDI comprising residue can be recirculated for agitating.

As a further alternative, it is also possible to recirculate TDI comprising residue through the solvent removal 3, the TDI removal 9 back into the storage vessel 23, if solvent is used for setting the viscosity, or through the TDI removal 9 back into the storage vessel 23, if the viscosity is set by the TDI content in the TDI comprising residue or by the temperature of the storage vessel.

After the process can be returned to normal operation, the TDI comprising residue stored in the storage vessel 23 is recycled into the process.

If for storing the TDI comprising residue a solvent is added, the solvent-diluted TDI comprising residue 27 is introduced into the solvent removal 3. Here, the solvent is removed from the solvent-diluted TDI comprising residue and the remaining TDI and high boilers are fed together with the crude TDI obtained from the TDI comprising crude reaction product into the TDI removal 9.

If for storing the TDI comprising residue TDI is added or the TDI removal is carried out in such a way that the TDI amount in the TDI comprising residue is such that it fulfils the viscosity conditions, the TDI-diluted TDI comprising residue 29 or the TDI comprising residue is fed into the TDI removal 9.

By recycling the TDI comprising residue, the solvent-diluted TDI comprising residue or the TDI-diluted TDI comprising residue, the yield can be increased because the amount of TDI removed from the process during a possible shutdown of the dryer or hydrolysis is minimized.

However, if the shutdown of the dryer or hydrolysis lasts for a period which is such that during continuing the workup process, the storage vessel 23 is completely filled with TDI comprising residue, TDI comprising residue which is formed after the storage vessel 23 is filled completely cannot be stored and must be withdrawn from the process. In this case, the TDI comprising residue may be introduced directly into the thermal use 21. Alternatively, if the TDI comprising residue further is fed into the storage vessel 23, the TDI comprising residue is withdrawn from the storage vessel 23 and introduced into the thermal use 21. In this case, the amount of TDI comprising residue withdrawn from the storage vessel 23 and introduced into the thermal use 21, preferably corresponds to the amount of TDI comprising residue which is fed into the storage vessel 23 from the TDI removal 9.

Further, also in case the stored TDI comprising residue in the storage vessel 23 has changed in such a way that a further use is not possible, this changed substance can be fed into the thermal use 21. This may be the case, for example, if the storage period exceeds a maximum storage period and the TDI in the TDI comprising residue polymerizes in such an amount that a reuse of the TDI comprising residue is not economical.

Withdrawing the TDI comprising residue either directly from the TDI removal 9 or from the storage vessel 23 and feeding it into the thermal use is shown by dashed lines in the FIGURE.

### EXAMPLES

A TDI comprising crude reaction product is worked up in two stages by removing solvent in a first distillation and removing TDI as product in a second distillation. In the second distillation, a TDI comprising residue is formed which has a NCO-number of 33.7 g/100 g and a viscosity of 120 mPas at 80° C. This TDI comprising residue is stored in a storage vessel.

The storage time for the TDI comprising residue with different additions of solvent or TDI is shown in table 1. The storage temperature kept stable at 80° C., the viscosity is measured at the same temperature and a shear rate of 250 s⁻¹ by means of a rotational viscometer.

In the comparative example, no solvent or TDI is added and the TDI comprising residue is stored as withdrawn from the second distillation.

In example 1, the TDI comprising residue is mixed with chlorobenzene as solvent in a ratio of TDI comprising residue:chlorobenzene of 1:1, in example 2, the TDI comprising residue is mixed with chlorobenzene as solvent in a ratio of TDI comprising residue:chlorobenzene of 2:1 and in example 3, the TDI comprising residue is mixed with chlorobenzene as solvent in a ratio of TDI comprising residue:chlorobenzene of 4:1. In example 4, the TDI comprising residue is mixed with TDI in a ratio of TDI comprising residue:TDI of 1:1.

TABLE 1
Viscosity increase

| | | Storage time (days) | | | | |
|---|---|---|---|---|---|---|
| | | 0 | 1 | 5 | 7 | 10 |
| Comparative | Viscosity (mPas) at 80° C., 250 s⁻¹ | 120.5 | 223 | 1537 | — | — |
| Example | | | | | | |
| Example 1 | Viscosity (mPas) at 80° C., 250 s⁻¹ | 9.3 | 9.3 | 10.7 | 12.9 | 15.0 |
| Example 2 | Viscosity (mPas) at 80° C., 250 s⁻¹ | 12.9 | 15 | 49 | 58.7 | 107 |
| Example 3 | Viscosity (mPas) at 80° C., 250 s⁻¹ | 27 | 30.4 | 665 | 1260 | — |
| Example 4 | Viscosity (mPas) at 80° C., 250 s⁻¹ | | 17.2 | 32.2 | 48.0 | 60.1 |

In the comparative example the viscosity increase was such that after a storage time of 3 days, the viscosity exceeded 1000 mPas and thus was so viscous that a further use was not possible.

The larger the amount of solvent which is mixed into the TDI comprising residue, the longer the diluted TDI comprising residue can be stored. In example 2, even after a storage time of 20 days the viscosity was below 1000 mPas and in example 1 the viscosity did not reach 1000 mPas even after 50 days.

The stored and diluted TDI comprising residue can be recycled into the workup process for the TDI comprising crude reaction product.

Besides adding a solvent or TDI, the viscosity also can be kept below 1000 mPas for several days and the storage period can be further increased, if the storage temperature is reduced.

Table 2 shows the viscosity increase depending on the storage time. The mixture used in examples 5 to 7 corresponds to the mixture of example 4, comprising the TDI comprising residue having the same composition as in examples 1 to 4, mixed with TDI in a ratio of 1:1.

In example 5, the mixture of TDI comprising residue and TDI was stored at a temperature of 40° C., in example 6 at a temperature of 60° C. and in example 7 at a temperature of 80° C. The viscosity was determined at the storage temperature, respectively, using the same method as described above.

TABLE 2
Viscosity increase depending on the storage temperature

| | | Storage time (days) | | | | |
|---|---|---|---|---|---|---|
| | | 1 | 5 | 10 | 15 | 20 |
| Example 5 | Viscosity (mPas) at 40° C., 250 s⁻¹ | 35.8 | 40.8 | — | 58.7 | 69.4 |
| Example 6 | Viscosity (mPas) at 60° C., 250 s⁻¹ | 23.7 | 33.6 | — | 74.5 | 95.2 |
| Example 7 | Viscosity (mPas) at 80° C., 250 s⁻¹ | 17.2 | 32.2 | 60.1 | 99.5 | 113 |

By reducing the storage temperature, also the storage time can be increased. As can be seen in table 2, example 7, even at a storage temperature of 80° C. it is possible to store the TDI comprising residue far more than 20 days before the viscosity reaches the limit of 1000 mPas.

1.-15. (canceled)

16. A process for storing a toluene diisocyanate and high boilers containing mixture, which contains 20 to 80 wt % toluene diisocyanate, the process comprising:
(a) feeding the toluene diisocyanate and high boilers containing mixture into a storage vessel (23); and
(b) agitating the toluene diisocyanate and high boilers containing mixture.

17. The process according to claim 16, wherein the viscosity of the toluene diisocyanate and high boilers containing mixture is set such that after a predefined storage time the viscosity is below 1 Pas.

18. The process according to claim 17, wherein the viscosity of the toluene diisocyanate and high boilers containing mixture is set by the content of solvent or toluene diisocyanate in the toluene diisocyanate and high boilers containing mixture or by setting the temperature.

19. The process according to claim 16, wherein the toluene diisocyanate and high boilers containing mixture is agitated by using a recirculation circuit through which the stored toluene diisocyanate is passed or by using an agitator in the storage vessel.

20. The process according to claim 16, wherein the toluene diisocyanate and high boilers containing mixture is a toluene diisocyanate comprising residue obtained by working up a toluene diisocyanate comprising crude reaction product.

21. A process for working up a toluene diisocyanate comprising crude reaction product, comprising:
(a) removing solvents from the toluene diisocyanate comprising crude reaction product in a solvent removal, thereby obtaining a crude toluene diisocyanate;
(b) removing toluene diisocyanate from the crude toluene diisocyanate in a toluene diisocyanate removal, thereby obtaining purified toluene diisocyanate as a product and a toluene diisocyanate comprising residue;
(c) feeding the toluene diisocyanate comprising residue into a dryer in which an essentially toluene diisocyanate comprising stream and a dryer residue are obtained and recycling the essentially toluene diisocyanate comprising stream into the toluene diisocyanate removal in step (b), or feeding the toluene diisocyanate comprising residue into a hydrolysis in which the toluene diisocyanate is hydrolysed, thereby forming the respective toluene diamine;
wherein at least part of the toluene diisocyanate comprising residue is stored in a storage vessel.

22. The process according to claim 21, wherein the toluene diisocyanate comprising residue is fed into the storage vessel when the dryer or hydrolysis is not operating.

23. The process according to claim 21, wherein the viscosity of the at least part of the toluene diisocyanate comprising residue which is stored in the storage vessel is set such that after a predefined storage time the viscosity is below 1 Pas.

24. The process according to claim 23, wherein the viscosity of the at least part of the toluene diisocyanate comprising residue is set by the content of solvent or by the content of toluene diisocyanate in the toluene diisocyanate comprising residue.

25. The process according to claim 24, wherein the solvent is selected from the group consisting of chlorinated aromatic hydrocarbons, aromatic or aliphatic hydrocarbons, ketones, esters, nitriles, sulfolane and mixtures thereof.

26. The process according to claim 23, wherein the viscosity of the at least part of the toluene diisocyanate comprising residue is set by setting the temperature in the storage vessel.

27. The process according to claim 23, wherein the viscosity of the at least part of the toluene diisocyanate comprising residue is set by setting the amount of toluene diisocyanate in the toluene diisocyanate comprising residue by the removing of toluene diisocyanate in (b).

28. The process according to claim 21, wherein the toluene diisocyanate comprising residue in the storage vessel is agitated.

29. The process according to claim 28, wherein for agitating, the storage vessel is connected to a recirculation circuit through which the stored toluene diisocyanate comprising residue is passed.

30. The process according to claim 28, wherein for agitating, the storage vessel comprises an agitator for stirring the toluene diisocyanate comprising residue in the storage vessel.
